(19) United States
(12) Patent Application Publication
(10) Pub. No.: US 2024/0041912 A1
(43) Pub. Date: Feb. 8, 2024

(54) COMPOSITION FOR TREATING A PATIENT SUFFERING FROM ULCERATIVE COLITIS, AND USE OF THE COMPOSITION AS A DRUG

(71) Applicant: Sterna Biologicals GmbH & Co. KG, Marburg (DE)

(72) Inventor: Jonas Renz, Munich (DE)

(21) Appl. No.: 18/488,067

(22) Filed: Oct. 17, 2023

Related U.S. Application Data
(63) Continuation of application No. 16/962,849, filed on Jul. 17, 2020, now abandoned.

(51) Int. Cl. A61K 31/711; C12N 15/113

(52) U.S. Cl. CPC A61K 31/711; C12N 15/1136; C12N 2310/127

(57) ABSTRACT

The invention relates to a composition for treating a patient suffering from a bowel disease associated with chronic inflammations, the composition comprising at least one DNAzyme which specifically inhibits the expression of GATA-3. A further aspect of the invention relates to the use of such a composition as a drug.

### CROSS-REFERENCE TO RELATED APPLICATIONS

This application is a United States National Stage Application filed under 35 U.S.C. § 371 claiming priority to and the benefit of PCT Application No. PCT/EP2019/050923, filed on Jan. 15, 2019, which claims priority to and the benefit of European Application No. 18152422.4, filed on Jan. 18, 2018, all of which are incorporated herein by reference in their entireties.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### SEQUENCE LISTING

The instant application contains a Sequence Listing submitted via EFS-Web and is hereby incorporated by reference in its entirety. Said ASCII copy, created on Sep. 27, 2022, is named SCR19P02US1_2022-09-27_sequence_listing_ST25 and is 918 bytes in size.

### STATEMENT REGARDING PRIOR DISCLOSURES BY THE INVENTOR OR A JOINT INVENTOR

Not Applicable

### BACKGROUND OF THE INVENTION

### Field of the Invention

The invention relates to a composition for treating a patient suffering from a bowel disease associated with chronic inflammations as claimed in claim 1 and to the use of such a composition as a drug as claimed in claim 9.

Unless otherwise indicated, the terms “expression” and “gene expression” are used synonymously below.

### Description of Related Art

Chronic inflammations are of great significance in medicine. Accordingly, it is important to find therapies for diseases such as, for example, bowel diseases associated with chronic inflammations. A known example of such a bowel disease is ulcerative colitis.

Besides Crohn's disease, ulcerative colitis is one of the most commonly occurring idiopathic and inflammation-dependent bowel diseases worldwide. It is characterized by an inflammatory attack of the large intestine or colon, which—unlike in the case of Crohn's disease for instance—is restricted only to the large intestine, where it is restricted only to the intestinal mucosa.

The genesis of chronic inflammatory reactions and of autoimmune diseases (which are often also inflammation-dependent) involves, inter alia, two transcription factors: the Th1 cell-specific transcription factor Tbet and the Th2 cell-specific transcription factor GATA-3. In this connection, Tbet induces the specific development of Th1 cells; by contrast, GATA-3 induces the specific development of Th2 cells. The balance between Th1 cells and Th2 cells is thus shifted in favor of the Th2 cells by the expression of GATA-3 and/or by the inhibition of Tbet. Analogously, the inhibition of GATA-3 and/or the expression of Tbet ensures a rising Th1 cell level. Ulcerative colitis is, for example, based on inflamed regions of the large intestinal mucosa, the GATA-3 mRNA levels of which are significantly increased (=increased GATA-3 expression) and which are thus Th2-dependent.

Numerous scientific publications have been published in relation to the specific equilibrium between GATA-3 and Tbet or between Th1 cells and Th2 cells. Consequently, attempts have been made in recent years to specifically inhibit or “switch off” the transcription factors GATA-3 and Tbet. One way of accomplishing this is the inhibition of the gene expression of these two transcription factors, modern gene technology providing various “tools” for this purpose. One of these tools includes DNAzymes.

DNAzymes represent a comparatively new class of antisense molecules. Antisense molecules refer to molecules which are able to bind single-stranded nucleic acids (generally mRNA). At the same time, a peculiarity of DNAzymes is that they exhibit not only this binding function, but also a catalytic function. They are thus capable of specifically cleaving single-stranded target DNA or target RNA and of thus degrading them (Sel et al. 2008). In this case, reference is also made to post-transcriptional inhibition because the inhibition of gene expression takes place at the mRNA level, i.e., even before the translation of the mRNA into a protein can follow.

Usually, DNAzymes of the 10-23 type are used for this purpose (Sontoro et al., 1997). Such DNAzymes have a catalytic domain of 15 nucleotides which is flanked by two substrate-binding domains. Said catalytic domain can comprise especially the conserved sequence ggctagctacaacga (SEQ ID No. 2). The specified sequence ggctagctacaacga (SEQ ID No. 2) is merely a preferred embodiment. A person skilled in the art is aware that DNAzymes of the 10-23 type having a modified catalytic domain can have a comparable biological activity. By contrast, the length of the substrate-binding domains is variable, it also being possible for two corresponding substrate-binding domains to differ from one another. In a preferred embodiment, the length of the substrate-binding domains is between 6 and 14 nucleotides, particular preference being given to a length of 9 nucleotides. Such DNAzymes have a specific sequence such as, for example, nnnnnnnnnggctagctacaacgannnnnnnnn (SEQ ID No. 3). Generally, the substrate-binding domains are completely complementary to the regions flanking the intended cleavage site of the target mRNA—to bind and cleave the target RNA, it is, however, not absolutely necessary for the DNAzyme to be completely complementary. DNAzymes of the 10-23 type cleave the target mRNA at sequences containing purine and pyrimidine in sequence. DNAzymes are exclusively chemically produced and are therefore not considered to be biological active ingredients.

DNAzymes are just as known for their use for specifically inhibiting the expression of GATA-3 or Tbet. For example, WO 2005/033314 A2 describes the mode of action of various DNAzymes. The disclosure content of WO 2005/033314 is considered to be the technological background of the present invention.

Problems which can occur when using DNAzymes as active ingredient are often attributable to the comparatively high instability and the sensitivity of nucleic acids. If they are not present in physiologically favorable solutions, nucleic acids tend to degrade rapidly, for instance by enzymatic degradation or physical stress—this applies especially to single-stranded nucleic acids, including DNAzymes as well. Furthermore, DNAzymes can differ in their specificity and thus in their effectiveness as active ingredient. Furthermore, the stability of DNAzymes can be impaired especially by changes in the pH of the DNAzyme-containing solutions. For example, a low pH (acidic environment) can result in hydrolysis of the DNAzyme molecules. An excessively high pH (basic environment) as well can influence the stability and thus the functionality of the DNAzymes, for instance by altering secondary structures of the molecules.

Common treatment strategies to date for patients with bowel diseases such as, for example, ulcerative colitis are determined by activity, occurrence (proctitis, left-sided colitis and extensive colitis) and pattern of the disease (disease course, response to previous medication, possible adverse effects of the therapies, etc.). Further factors that significantly influence the question of the therapeutic decision are duration of the disease, age at the start and accompanying diseases of the patient. The activity of the disease is also an important indicator of the need to admit the patient to a hospital.

Generally, patients suffering from ulcerative colitis are first treated with mesalazine (5-aminosalicylic acid), which is rectally and orally administrable. If the symptoms of the disease persist thereafter, corticosteroids are commonly administered, and patients with severe ulcerative colitis often need to be treated in hospital to intensify the corticosteroid therapy. If even the corticosteroids do not work and do not result in any improvement in the patient's status, what can be carried out as the current last-resort option is a calcineurin-inhibiting therapy, for instance using ciclosporin or tacrolimus or using monoclonal antibodies directed against TNF for example.

Nevertheless, none of the aforementioned therapeutic methods is suitable for treating bowel diseases associated with inflammations specifically. On the contrary, the substances used are known for their highly immunosuppressant action. Furthermore, the substances are associated with commonly occurring adverse effects, such as, for example, increased immunosuppression, hepatotoxicity, myocardial hypertrophy, gastrointestinal complaints, fatigue and dizziness, diarrhea, emesis, nephrotoxicity, neurotoxicity, tremor, hypertension, insomnia, depressions, cramps, masking of infections, hyperkalemia, hyperglycemia, increased risk of tumors—to name but just a few.

It is thus an object of the invention to eliminate the disadvantages in the prior art and to provide a composition suitable for treating patients suffering from a bowel disease associated with chronic inflammations. In particular, what is to be provided in this connection is an active ingredient which is suitable for effectively treating ulcerative colitis without leading to considerable secondary complaints at the same time. Furthermore, what is to be ensured is an improved remission.

### BRIEF SUMMARY OF THE INVENTION

According to the invention, the object is achieved by a composition for treating a patient suffering from a bowel disease associated with chronic inflammations, the composition comprising at least one DNAzyme which specifically inhibits the expression of GATA-3. The specific DNAzymes bind and cleave in vivo the mRNA of the transcription factor GATA-3, which, as a protein, has a central role in the genesis of Th2-dependent chronic inflammatory diseases. As a result of this cleavage, the mRNA can no longer be translated into a functional protein. Consequently, the GATA-3 protein level is significantly minimized.

### BRIEF DESCRIPTION OF THE SEVERAL VIEWS OF THE DRAWINGS

FIG. 1 shows mean values of total MAYO scores from a series of experiments to test the composition according to the invention in a graph;

FIG. 2 shows mean values of total MAYO scores from the series of experiments from FIG. 1 in a table;

FIG. 3 shows mean values of sigmoid endoscopic MAYO scores from a series of experiments to test the composition according to the invention as a bar chart; and

FIG. 4 shows mean values of endoscopic MAYO scores from the series of experiments from FIG. 3 in a table.

### DETAILED DESCRIPTION OF THE INVENTION

A person skilled in the art is aware that downregulation of expression can be understood to mean either a partial inhibition or a complete inhibition of the expression. In any case, the expression is significantly reduced in comparison with the natural expression level.

In this connection, what can be provided according to a preferred embodiment is that the DNAzyme has the sequence hgd40 (GTGGATGGAggctagctacaacgaGTCTTGGAG) (SEQ ID No. 1). This sequence exhibits particularly high enzyme activity and cleaves GATA-3 mRNA with very high specificity and with high efficiency. Consequently, a DNAzyme having the sequence hgd40 is outstandingly suitable for specific and effective treatment of bowel diseases which are Th2-dependent, i.e., which correspond to an increased GATA-3 level. However, DNAzymes having other sequences with which the expression of GATA-3 can likewise be specifically inhibited are also conceivable.

According to an advantageous embodiment of the invention, what is provided is that the concentration of the DNAzyme in the composition is between 0.75 mg/ml and 75 mg/ml. What is especially provided in this connection is that the concentration of the DNAzyme in the composition is approx. 7.5 mg/ml. Such a concentration proves to be particularly efficient. DNAzyme concentrations of under 0.75 mg/ml may be associated with a high loss of action of the composition. At the same time, the risk of toxicity, which may exist when administering the composition, may increase in the case of concentrations of over 75 mg/ml. With respect to absolute amounts, what can be provided according to the invention is that the DNAzyme hgd40 is administered in a dose of from 10 mg to 500 mg per patient per day. What can be especially provided is a dose which is between 50 mg and 250 mg per patient per day. Surprisingly, such a composition has the result that a positive effect of the active ingredient can also still be observed weeks after administration of the composition. The composition is therefore outstandingly suitable for treating a patient suffering from a bowel disease associated with chronic inflammations because DNAzymes administered in this way display their action over a long period of time. The composition according to the invention thus leads to a high degree of remission in the patients treated with the composition.

It is also advantageous when the composition is in the form of an aqueous solution. Producing such a solution is economical and simple. In addition, an aqueous solution does not have increased toxicity, meaning that it is suitable for use in therapeutics.

A further advantage arises from the design variant according to which the composition comprises at least one salt. What is especially provided in this connection is that the salt is sodium chloride and/or potassium chloride and/or a phosphate. According to the invention, it is intended that the hgd40 be present in a dissolved state in PBS for use, and so the composition is essentially a phosphate-buffered saline solution (PBS) containing the DNAzyme as active ingredient. However, other buffers common in biochemistry are also conceivable. In addition, further additives such as, for example, EDTA can be added.

According to an advantageous further development of the invention, what is provided is that the composition is suitable for rectal administration. This has the advantage that the DNAzyme active ingredient hgd40 can act directly at the site of inflammation, namely at the surface mucosa of the large intestine. There, active ingredient is taken up by the inflamed mucosa. The result is a rapid and efficient action of the therapeutic. However, oral forms of administration such as, for example, capsules, pills or tablets are also conceivable.

As a further feature of the composition, what can be provided is that the composition is, in the case of a desired therapy period of n days, administered over an administration period of not more than n days. What can be intended in this connection is that the administration period comprises, in the case of a desired therapy period of n days, not more than

$$\frac{3 n}{4}$$

days of the desired therapy period. What can be especially provided is an administration period of not more than

$$\frac{n}{2}$$

days of the desired therapy period. Said therapy period is to be understood to mean the period during which a patient suffering from a bowel disease associated with chronic inflammations is to be treated (i.e., during which the active ingredient of the composition is to act in the patient and therefore control the disease symptoms).

Such a dosing scheme, in which the composition is, in the case of a desired therapy period of n days, to be administered over an administration period of not more than n days, is possible not least because the composition leads to a high degree of remission in the treated patients. This in turn results in the advantageous effect of a patient not having to be treated longer than necessary. This is desirable because it provides relief for the patient to be treated.

A further aspect of the invention consists in the use of the composition as a drug for treating a patient suffering from a bowel disease associated with chronic inflammations. The composition can also be referred to as SB012 when it is used as a drug. What can be especially provided in this connection by the invention is that the composition is administered in the form of a suppository. Alternatively, what can be provided is that the composition is administered in the form of an enema or a rectal foam. Advantageously, the composition SB012 can thus be used to treat affected patients either as a drug within a supportive therapy or in the course of a therapy of its own. What especially arises, then, from the form of the composition or the drug as suppository, enema or rectal foam is a simple and rapid administrability of the drug. Consequently, the drug can be administered on an outpatient basis and, if necessary, even without the help of skilled staff.

In a preferred use variant, the composition is administered such that the administered dose of the DNAzyme is between 10 mg and 500 mg per patient per day. What can be especially provided in this connection is a dose which is between 50 mg and 250 mg per patient per day. Surprisingly, such use of the composition has the result that a positive effect of the active ingredient can also still be observed weeks after administration of the composition. Said use is therefore outstandingly suitable for treating a patient suffering from a bowel disease associated with chronic inflammations because DNAzymes administered in this way display their action over a long period of time. The preferred use thus leads to a high degree of remission in the patients treated with the composition.

In addition, what can be provided in the use according to the invention is that the composition is, in the case of a desired therapy period of n days, administered over an administration period of not more than n days. What can be intended in this connection is that the administration period comprises, in the case of a desired therapy period of n days, not more than

$$\frac{3 n}{4}$$

days of the therapy period. What can be especially provided is an administration period of not more than

$$\frac{n}{2}$$

days of the desired therapy period. The reason for this is the high degree of remission of the composition. A drug administered according to such a dosing scheme, i.e., in which the drug is, in the case of a desired therapy period of n days, administered over an administration period of preferably not more than

$$\frac{n}{2}$$

days, has the positive effect of a patient suffering from a bowel disease associated with chronic inflammations not having to be treated longer than necessary. A shorter administration period minimizes the stress on the patients to be treated and is therefore advantageous.

The above-described advantages and effects arise especially when using the composition according to the invention in the treatment of a patient suffering from a bowel disease associated with chronic inflammations.

Further features, details and advantages of the invention become apparent from the wording of the claims and from the following description of exemplary embodiments and figures.

In one embodiment, the composition containing the DNAzyme hgd40 is present in a pharmacologically compatible carrier together with excipients and/or fillers or as a solvent.

The composition can then, for example, be produced and administered in the form of suppositories, drops, mouth spray, nasal spray, pills, tablets, film-coated tablets, layered tablets, suppositories, gels, ointments, syrup, powders for inhalation, granules, emulsions, dispersions, microcapsules, capsules, powders, or solutions for injection. Moreover, the group of pharmacologically compatible carriers encompasses formulations such as layered tablets for the controlled and/or continuous release of the active ingredient and also microencapsulations as a specific dosage form.

Such formulations are highly suited for rectal administration. However, other forms of administration can also be provided, such as, for example, inhalation or intravenous, intraperitoneal, intramuscular, subcutaneous, mucocutaneous, oral, transdermal, topical, buccal, intradermal, intragastric, intracutaneous, intranasal, intrabuccal, percutaneous or sublingual administration.

The addition of excipients improves the pharmaceutical properties of the composition for the specific use. Examples of pharmacologically compatible excipients that can be used are lactose, starch, sorbitol, sucrose, cellulose, magnesium stearate, dicalcium phosphate, calcium sulfate, talc, mannitol, ethyl alcohol and the like. The aforementioned carriers can consist of such an excipient to an extent of up to 95% (w/w). To prepare suppositories, preference is given to using low-melting waxes, fatty acid esters and/or glycerides.

When processing small to very small amounts, fillers are usually required, which ensure that the composition according to the invention receives the necessary size/mass and can be used without any problems. The filler used is a substance from the group comprising starches (corn, potato and wheat starch), lactose, glucose, mannitol, sorbitol and fructose.

Furthermore, disintegrants, colorants, flavorings and/or binders can be additionally added to the composition. The disintegrants used are substances from the group comprising starches (corn, potato and wheat starch, sodium carboxymethyl starch), natural and synthetic gums such as, for example, locust bean gum, karaya, guar, tragacanth, agar, cellulose derivatives such as methylcellulose, sodium carboxymethylcellulose, microcrystalline cellulose, alginates, aluminas, bentonites, PVP (polyvinylpyrrolidone), Carbopol and magnesium peroxide. These constituents can be used in amounts of up to 30% (w/w). Disintegrants ensure good compressibility of the composition by improving particle adhesion. They also facilitate later disintegration, for instance in the gastrointestinal tract. They work by absorbing moisture, increasing capillarity and swelling, or evolving gases and effervescing under the influence of moisture, or by increasing the wettability of the tablets as a hydrophilization agent. Binders ensure cohesion in granules and, alongside applied pressure, tablet strength.

Liquid formulations encompass solutions, suspensions, sprays and emulsions, such as, for example, aqueous solutions for injection or solutions based on water and propylene glycol for parenteral injections.

Lubricants can also be provided. Lubricants are an umbrella term for flow agents, lubrication agents and mold-release agents. Flow agents generally improve flow properties by reducing frictional and adhesive forces between particles of bulk materials. They reduce interparticle friction and reduce surface moisture.

The lubricants used can be substances from the group comprising boric acid, stearates (e.g., magnesium stearate, calcium stearate, potassium stearate), stearic acid, high-melting waxes and water-soluble substances from the group comprising sodium chloride, sodium benzoate, sodium acetate, sodium oleate, polyethylene glycol and amino acids (e.g., leucine). Lubricants can be used in amounts of up to 15% (w/w).

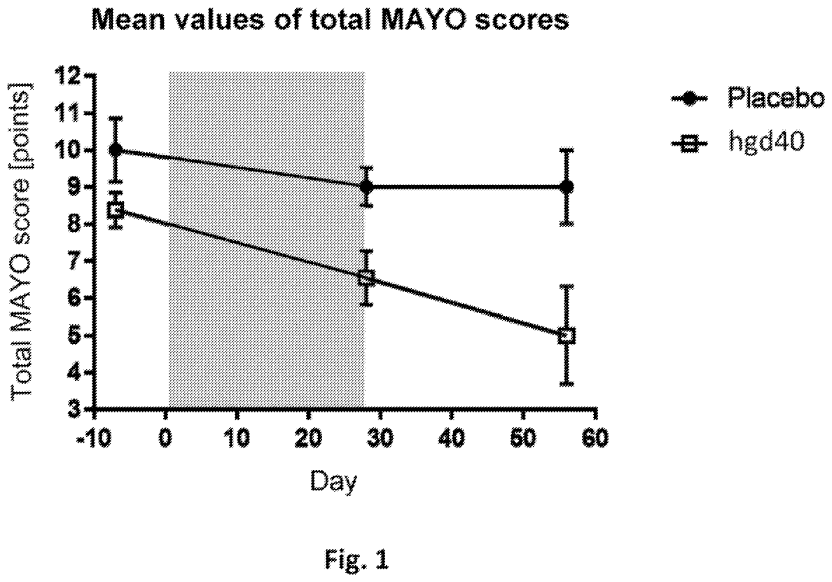

FIGS. 1 to 4 are each based on the same series of experiments. In the series of experiments, patients were treated with a DNAzyme which has the sequence GTGGATGGAggctagctacaacgaGTCTTGGAG (hgd40) (SEQ ID No. 1) and which specifically inhibits or downregulates the expression of GATA-3. However, treatment with other DNAzymes which specifically inhibit or downregulate the expression of GATA-3 is also conceivable. In parallel to the treatment with the DNAzyme hgd40, a PBS buffer solution was administered to a control group. Said control group consequently did not receive a DNAzyme.

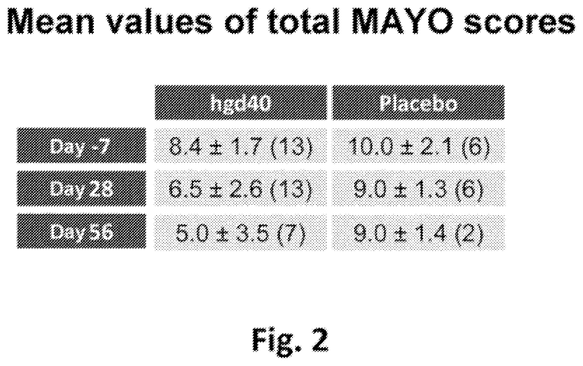

FIG. 1 and FIG. 2 both depict the same results. In FIG. 1, mean values of total MAYO scores from the aforementioned series of experiments to test the composition according to the invention are shown in a graph. In FIG. 2, mean values of total MAYO scores from the series of experiments from FIG. 1 are depicted in a table. The MAYO score is used especially in clinical studies as an index for determining the disease activity of ulcerative colitis. This four-level index includes the stool frequency, the severity of rectal bleedings, the endoscopic assessment of the mucosa and the overall assessment by a physician. In the graph, the mean value of the MAYO scores of patients from the series of experiments (Y-axis) is plotted against the duration of treatment in days (X-axis).

In the series of experiments that corresponds to the MAYO scores shown in FIG. 1 and FIG. 2, patients were treated with a DNAzyme which has the sequence GTGGATGGAggctagctacaacgaGTCTTGGAG (SEQ ID No. 1) and which specifically inhibits or downregulates the expression of GATA-3 (FIGS. 1 and 2, hgd40). In parallel to the treatment with the DNAzyme hgd40, a PBS buffer solution was administered to a control group (FIGS. 1 and 2, Placebo). The control group consequently did not receive a DNAzyme. The period in which the patients and the control group were treated with the active ingredient and with the placebo, respectively, is highlighted in gray in FIG. 1 (28 days). The MAYO scores were measured three times: 7 days before the start of the administration period (−7), 28 days after the start of the administration period (28) and 28 days after the end of the administration period (56).

In FIG. 1, the MAYO scores in the table from FIG. 2 are depicted as a graph. What becomes impressively apparent here is that the mean value of the MAYO scores of the patients who were treated with hgd40 decreases to a significantly higher extent than the mean value of the MAYO scores from the control group. If, at the start of the series of experiments (−7), the mean MAYO score of the treated patients is still approx. 8.4, it is, 28 days after the start of the administration period, already approx. 6.5. At the end of the series of experiments (56), the mean MAYO score of the patients treated with hgd40 is approx. 5.0 (cf. FIG. 2). Overall, a decrease in the mean MAYO score of the patients of approx. 3.4 can thus be observed. In comparison, the mean MAYO score of the control group only decreases by approx. 1.0 (from 10.0 to 9.0; FIG. 2).

The results depicted in FIG. 1 and FIG. 2 impressively demonstrate that the composition according to the invention is suitable for treating a patient suffering from a bowel disease associated with chronic inflammations.

What is particularly advantageous in this connection is that a positive effect of the active ingredient can also still be observed over a period of at least 28 days after the end of the last administration of the composition. This effect becomes apparent from the fact that the MAYO scores of the treated patients also still decrease after day 28 (last day of the administration period) over a period of at least four further weeks. The composition according to the invention therefore surprisingly leads to a high degree of remission in the treated patients.

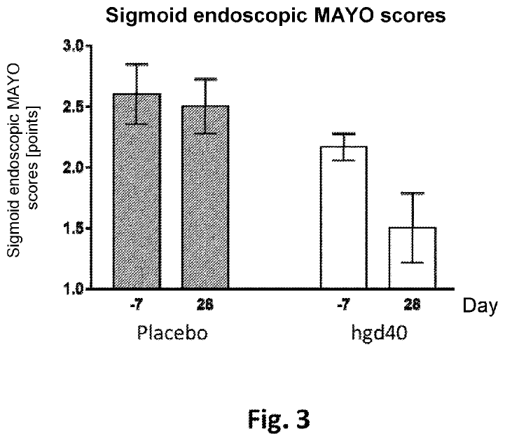

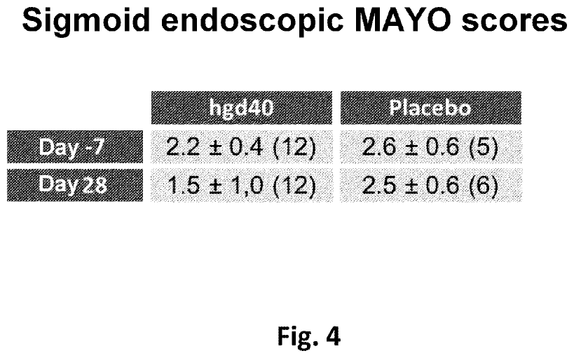

FIG. 3 and FIG. 4 are based on the same series of experiments as in FIG. 1 and FIG. 2. However, the results corresponding to FIG. 3 and FIG. 4 were obtained from other points of view. In FIG. 3, the mean values of sigmoid endoscopic MAYO scores are depicted as a bar chart. The associated measurement data can be seen in FIG. 4. A person skilled in the art understands the term “sigmoid” to mean the last part of the large intestine in humans, the colon sigmoideum. In the case of the endoscopic MAYO score, endoscopic findings are assessed with respect to disease-typical characteristics.

Analogously to FIGS. 1 and 2, in the series of experiments that corresponds to the endoscopic MAYO scores shown in FIG. 3 and FIG. 4, patients were treated with a DNAzyme which has the sequence GTGGATGGAggctagctacaacgaGTCTTGGAG (SEQ ID No. 1) and which specifically inhibits or downregulates the expression of GATA-3 (FIG. 3 and FIG. 4, “hgd40”). However, treatment with other DNAzymes which specifically inhibit or downregulate the expression of GATA-3 is also conceivable in the case of FIG. 3 and FIG. 4. In parallel to the treatment with the DNAzyme hgd40, PBS buffer solution was administered to a control group (FIG. 3 and FIG. 4, Placebo). Said control group consequently did not receive a DNAzyme. The period in which the patients and the control group were treated with the active ingredient and with the placebo, respectively, was 28 days. The MAYO scores were measured twice: 7 days before the start of the administration period (−7) and on the 28th and last day of the administration period (28).

The bar charts in FIG. 3 provide an overview of the overall course of the series of experiments. Depicted in dark gray are the bars corresponding to the endoscopic MAYO scores of the control group (FIG. 3, Placebo). The white bars in FIG. 3 correspond to the MAYO scores of the patients who were treated with hgd40 (FIG. 3, hgd40). In FIG. 3, what is then compared in each case is the mean value of the MAYO scores at the first time point (7 days before the start of the administration period;−7) with the mean value of the MAYO scores at the end of the administration period (28th day of the administration period; 28). No significant difference can be seen here in the case of the control group (cf. FIG. 3, gray bars). The mean value of the MAYO scores was approx. 2.6 on day−7 and approx. 2.5 on day 28 (cf. FIG. 4, Placebo).

This contrasts with the findings of the patients treated with hgd40. In the case of the patients treated with hgd40, MAYO scores were likewise given at the first time point (−7) and at the end of the administration period (28th day). The mean values of these MAYO scores are compared in FIG. 3 (FIG. 3, white bars). What can be clearly seen here in the group of patients treated with hgd40 is that there is a significant difference between the mean value of day−7 and the mean value of day 28. The mean MAYO score is thus reduced from approx. 2.2 (day−7) to approx. 1.5 (FIG. 4, hgd40).

The results depicted in FIGS. 1 to 4 thus impressively demonstrate that the composition according to the invention comprising at least one DNAzyme which specifically inhibits the expression of GATA-3 is suitable for treating a patient suffering from a bowel disease associated with chronic inflammations. In particular, the results show that the complaints which usually occur in connection with chronic inflammatory bowel diseases can be reduced effectively. At the same time, the results in FIGS. 1 and 2 especially show that the composition ensures improved remission.

1. A composition for treating a patient suffering from a bowel disease associated with chronic inflammations, the composition comprising at least one DNAzyme which specifically inhibits the expression of GATA-3.

2. The composition as claimed in claim 1, characterized in that the DNAzyme has the sequence hgd40 (GTGGATGGAggctagctacaacgaGTCTTGGAG).

3. The composition as claimed in either of claims 1 and 2, characterized in that the concentration of the DNAzyme in the composition is between 0.75 mg/ml and 75 mg/ml.

4. The composition as claimed in any of the preceding claims, characterized in that the composition is in the form of an aqueous solution.

5. The composition as claimed in any of the preceding claims, characterized in that the composition comprises at least one salt.

6. The composition as claimed in claim 5, characterized in that the salt is sodium chloride and/or potassium chloride and/or a phosphate.

7. The composition as claimed in any of the preceding claims, characterized in that the composition is suitable for rectal administration.

8. The composition as claimed in any of the preceding claims, characterized in that the composition is, in the case of a desired therapy period of n days, administered over an administration period of not more than n days, especially an administration period of not more than
$$\frac{n}{2}$$
days.

9. The use of the composition as claimed in any of claims 1 to 9 as a drug for treating a patient suffering from a bowel disease associated with chronic inflammations.

10. The use as claimed in claim 9, characterized in that the composition is administered in the form of a suppository.

11. The use as claimed in claim 10, characterized in that the composition is administered in the form of an enema and/or a rectal foam.

12. The use as claimed in any of claims 9 to 11, characterized in that the composition is administered such that the administered dose of the DNAzyme is between 10 mg and 500 mg per patient per day.

13. The use as claimed in any of claims 9 to 12, characterized in that the composition is, in the case of a desired therapy period of n days, administered over an administration period of not more than n days, especially an administration period of not more than
$$\frac{n}{2}$$
days.

14. The composition as claimed in any of claims 1 to 8 for use in the treatment of a patient suffering from a bowel disease associated with chronic inflammations.


SEQUENCE LISTING (1 sequence)

SEQ ID NO: 1 - DNA, 33 bp, Homo sapiens
  source 1..33: mol_type = other DNA
gtggatggaggctagctacaacgagtcttggag